(19) United States
(12) Patent Application Publication
(10) Pub. No.: US 2005/0192584 A1
(43) Pub. Date: Sep. 1, 2005

(54) Bone shaping device for knee replacement

(76) Inventors: Peter Stanley Walker, New York, NY (US); Chih-Shing Wei, Lattingtown, NY (US)

Correspondence Address: Stephen E. Feldman, P.C., Suite 701, 12 East 41st Street, New York, NY, 10017, US

(21) Appl. No.: 11/121,202

(22) Filed: May 4, 2005

Related U.S. Application Data
(62) Division of application No. 10/452,707, filed on May 30, 2003.

(51) Int. Cl. A61B 17/58

(52) U.S. Cl. 606/79

(57) ABSTRACT

A bone surgery device with a reciprocating cutting head. The device comprises several sets of shaping surfaces sometimes including cutting blades, which are located and oriented so as to shape the distal end of the femur and the proximal end of the tibia for total knee replacements and unicompartmental knee replacements. The device, which may be hand directed or aided by spatial and directional guiding mechanisms or an optical navigation system, may also include both coolant supply for controlling heat during bone cutting and shaping, and a suction method for carrying away fluid and debris. The device is substantially configured to the artificial joint component to be attached thereto.

### BACKGROUND OF THE INVENTION

1. The Field of the Invention

The present invention relates to a device for use in joint replacement surgery such as total knee replacement where the entire bearing surfaces of the distal femur and proximal tibia are replaced. The invention is also related to unicompartmental knee replacements where only the lateral or medial compartments are replaced. The purpose of the invention is to cut and shape the distal end of the femur and the proximal end of the tibia so that the artificial components to be installed will precisely and accurately fit on their respective bones. Use of the device during knee replacement surgery increases the accuracy with which the artificial components fit to the bones and achieves a more consistent overall alignment of the femur and the tibia. Use of the device reduces the time to cut and shape the bone surfaces and is consistent with so-called minimally-invasive surgery.

2. Background of the Invention

Currently, there are a number of manufacturers who produce at least one artificial knee replacement system of varying types in multiple sizes. Each of these replacement components typically utilizes a set of surgical instruments which are used in the preparation of the femur and tibia prior to receiving and implanting the artificial knee replacements. Some of the replacement systems have several sets of surgical instruments which are a response to the different alignment goals or preferences of the surgeon. These instrument sets have many independent jigs and fixtures, some of which have slots for passing through the blade of a reciprocating saw used to cut the bone. The jigs and fixtures have to cater to all of the various sizes and thicknesses of the knee replacement system components and accommodate all of the various bone cuts which are required to be made. A typical femoral component has a shape which requires five different cutting operations in order to properly interface with the bone. Accordingly, considerable training and experience is required for both the surgeon and assisting operating staff to become familiar with the varying instrument systems in order to achieve accurate and reproducible results without an extended operating time.

Some of the current knee replacement systems use more than one cutting guide for the five bone surfaces that must be cut and shaped. Due to the successive cuts that are made separately, there is the possibility of a resulting lack of accurate registration between the successive cuts. The problem of mis-registration is reduced when cement is utilized for bone to artificial component fixation, due to the filling nature of the cement. However, this mis-registration becomes more important when a boney ingrowth surface is used on the components. Under these circumstances, a more precise fit is required between the bone and artificial component which requires a considerable amount of surgical time and effort to set up the various instrument and cutting guides to achieve the accurate multi-surface registration. To attach some of the cutting guides, intramedullary rods are often employed, while the guides themselves are either pinned or screwed to the bones once their proper positions are achieved. Typically, the cutting guides are flat surfaces or slots across or through which a reciprocating saw is used to cut through the bone. Inaccuracies occur due to the number of cuts required, the flexibility of the saw blade, the looseness of the fit of the blade within the slot and the variations in hardness of the bone. These inaccuracies result in reduced implant-bone contact and diminished bone ingrowth or attachment. However, the present invention uses integral and rigid grinding surfaces which have the same shape as the implant. As a consequence the resulting surfaces shaped into the bone will be an accurate match.

Employment of the device of the present invention also enables the surgeon to reduce the invasiveness of the procedure due to use of a single cutting device and procedure to make a multiple faceted cut or a curved line cut at one time. Hence, compared to classical procedures currently employed in knee replacement surgery, the present invention should reduce the time of operation, achieve accurate component fit, improve fixation, and reduce soft tissue damage.

An alternate use of the bone shaping concept, rather than shaping external bone surfaces, is to cut a curved channel into the interior of the bone to receive an implant, such as for the femoral component of a hip replacement.

### SUMMARY OF THE INVENTION

The present invention overcomes the problems and disadvantages of the prior art by providing a single device for cutting and shaping each bone for its respective component in knee replacement surgery. The device utilizes modular and interchangeable shaping heads for varying sizes and shapes of replacement components. The shaping heads in their preferred embodiment are constructed having multiple shaping surfaces, sometimes with cutting blades. The shaping heads are interchangeable for conforming and adapting to the specific size and shape of knee replacement components.

The device of the present invention is either hand-held by the surgeon or can be supported by directing devices. The device of the preferred embodiment is formulated to be used in conjunction with knee replacement surgery and is configured to move the shaping head in a precise direction to shape and cut the upper portion of the tibia, or the distal portion of the femoral bone's multi-faceted elements simultaneously. For the tibia, the device of the present invention would include mainly a flat shaping surface due to the fact that the knee replacement tibia interface is flat in nature. For the femur, the device is positioned proximate the femoral bone to be cut and shaped, while the shaping head of the device is vibrated to perform a shaping operation, or a shaping and cutting operation, to the bone. The side-to-side reciprocating motion of the shaping head needs to be sufficient only to create a small motion such as 1-5 mm to the shaping surfaces. In cutting the different facets of the distal femur, the cuts which are essentially vertical, notably at the anterior and posterior, can be cut using the integral cutting blades while the other facets are cut using shaping surfaces. Alternatively, broad shaping surfaces can be used for these anterior and posterior cuts. Further, a cavity can also be cut into the interior of the femur for the femoral component of a hip prosthesis.

The cutting blades are saw-like in shape and function and are intended to engage the bone to be shaped and cut prior to the engagement of the shaping surfaces. The shaping surfaces are meant to engage the bone to be shaped subsequent to the cutting blades and resemble a rasp-like surface which, through the vibratory movements of the device, grind down the bone to its final and intended configuration. The extent of the vibration is sufficient to reciprocate the cutting blades in a side-to-side manner and, at a short time later, to provide the necessary reciprocating motion to the rasp-like shaping surfaces for shaping and configuring the other portions of the bone.

Using the above described methodology, there is a significant reduction in the time taken to cut and shape the bone surfaces. There is also realized an increase in the accuracy and overall consistency in the alignment of the knee replacement to the bone axes and of the bone axes to each other. Precision and accuracy in cutting and shaping the bone interfacing with the knee replacement components permits more reliable bony ingrowth to bind with the component, as an alternate to employing a cement fixation. The alignment to the bone axes can be achieved using alignment devices such as rods attached to the bone surgery device. Alternatively, optical or other navigation systems can be used. In addition to the components of the invention just described, the device can include both a means for supplying coolant to the shaping heads and a means for suctioning away the spent coolant and bone particles created by the shaping operation. As a result, there is realized a clean and temperature-reduced cutting area.

Additional objects and advantages of the present invention will be set forth, in part, in the description which follows, and will in part be obvious from the description, or may be learned by practice of the invention. A particular aspect is that such a bone surgery device can be used for the shaping of bones other than at the knee. One example is the femoral cavity for locating a hip replacement. Other examples include surfaces and cavities for the other joints of the body. The objects and advantages of the invention may be realized and attained by means of the instrumentalities and combinations particularly pointed out in the appended claims.

### BRIEF DESCRIPTION OF THE DRAWINGS

For a further understanding of the nature and objects of the present invention, reference should be made to the following detailed descriptions, taken in conjunction with accompanying drawings, in which like parts are given like reference numerals, and wherein:

FIG. 1 is a partial perspective view of the preferred embodiment of the apparatus of the present invention for shaping the distal femur for a total knee replacement.

FIG. 1a is a side view of the lower limb around the knee joint showing the present invention being introduced to the knee.

FIG. 2 is a view similar to that of FIG. 1 but with one side of the casing removed.

FIG. 3 is a partial perspective showing the mechanism to produce side-to-side reciprocating motion to the shaping head.

FIG. 4 is a partial side view of the cutting and shaping device in position against the distal femur at the initiation of the shaping operation.

FIG. 5 is a partial side view of the femur and the cutting and shaping device after the shaping operation is completed.

FIG. 6 is a side view showing the replacement of the femoral component of a total knee replacement on to the distal femur.

FIG. 7 shows the apparatus of the invention with a fluid cooling and suction means.

FIG. 8 shows some alternate forms of the cutting and shaping heads.

FIG. 9 shows some of the shapes of total and unicompartmental knee replacements which can be cut and shaped using versions of the apparatus.

FIG. 10 shows the apparatus of the invention with an alignment means consisting of an external rod.

FIG. 11 shows the apparatus of the invention with an alignment means consisting of an intramedullary rod.

FIG. 12 shows the apparatus of the invention with an alignment means consisting of an optical navigation system.

FIG. 13 shows an alternate form of the embodiment shown in FIG. 1, where the apparatus is built around a pre-exiting drill unit.

FIG. 14 shows the device for generating reciprocating motion, with a shaping device attached in the specific direction for shaping the medullary canal of the femur for a hip replacement.

FIG. 15 shows an alternative form of the invention where a cavity is cut into the proximal femur.

### DESCRIPTION OF THE PREFERRED EMBODIMENT

Reference will now be made in detail to the preferred embodiment of the present invention, an example of which is illustrated in the accompanying drawings.

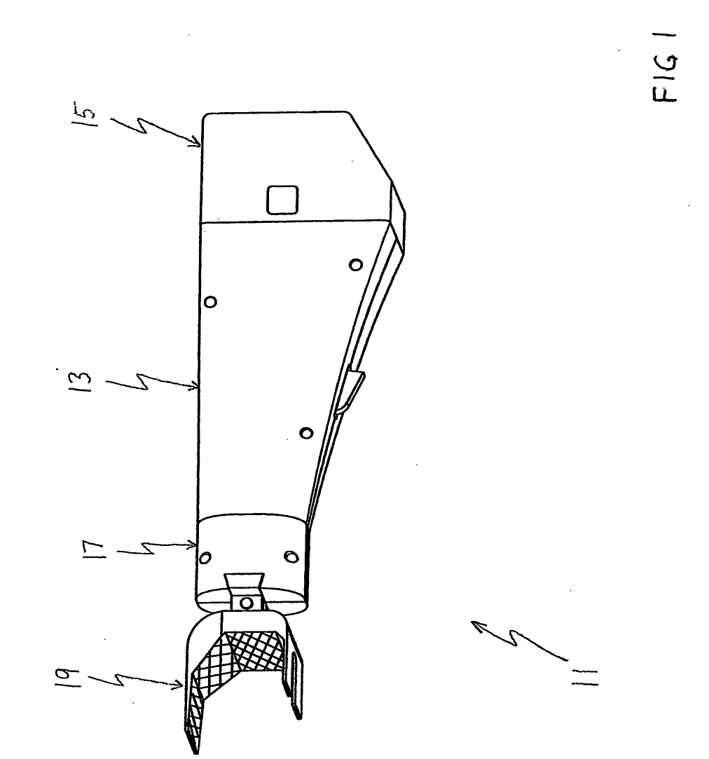

The preferred embodiment of the present invention is illustrated at 11 in FIG. 1. The device is adapted for cutting and shaping during use on the distal femur. The surgery is normally performed with the patient lying down and with the knee flexed at an angle of about 110 degrees as shown in FIG. 1a.

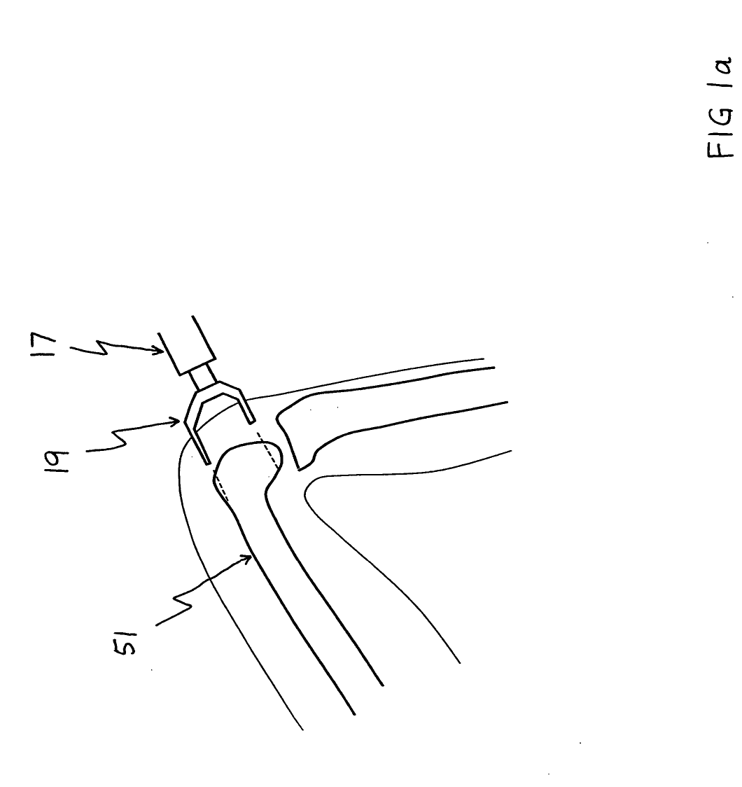

The device shown consists of a self-powered unit which is an advantage for use in the operating room. The main part of the outer casing 13 contains the motor 25 and hand switch 23 as shown in FIG. 2. The outer casing 13 also functions as a handle for the device. The end of the casing 15 contains the battery pack. The front of the casing 17 contains the mechanism for converting the rotary motion of the motor to side-to-side reciprocating motion. The shaping head 19 is attached to the end of the mechanism and hence the motion is transmitted there. Further details of the assembly are shown in FIG. 2, where the outer casing 13 is removed. The power switch box 21 and switch 23 enable the surgeon to control the motion and speed. The motor 25 is powered by the battery located in 15. The output-rotating shaft is fitted with an offset track roller 27 which engages in a slot in slotted slider 29. This mechanism causes the rotation of the motor to be converted to side-to-side reciprocating motion which is transmitted to the shaping head 19. The shaping head is attached to the slotted slider 29 by a grooved clevis pin and retaining ring combination 30.

A dovetail-shaped sliding arm 32 of the slotted slider 29 rides on two similarly dovetail-shaped shoulder openings 34 at the outer edge of the front of the casing 17. To reduce friction and wear between the sliding arm 32 and the slot of the casing 34, their interface areas may be lined with a polymeric material such as teflon or high molecular weight polyethylene.

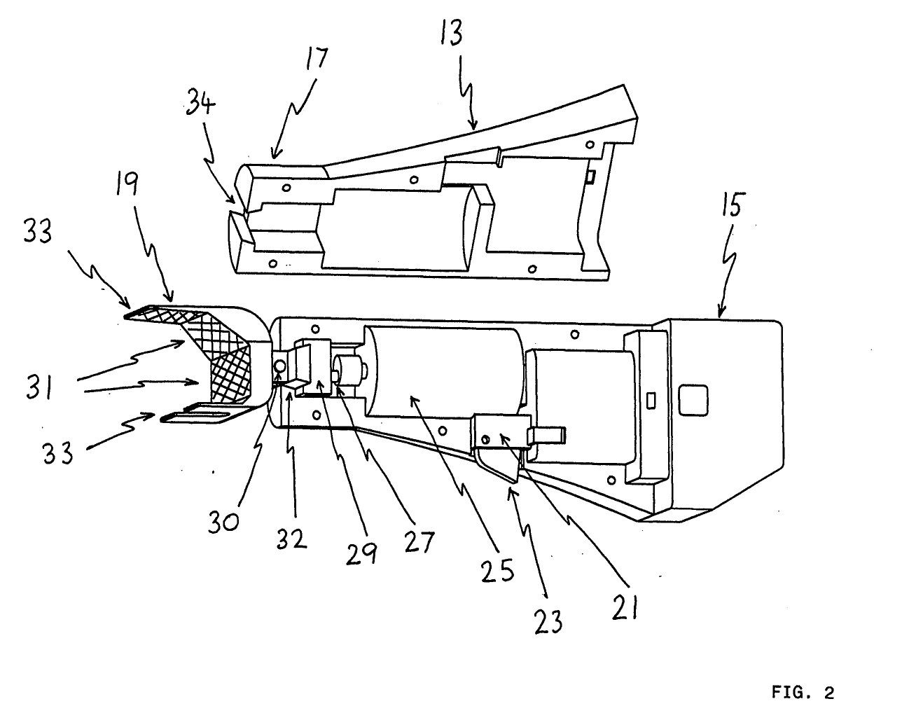

The shaping head itself comprises shaping or rasping surfaces 31, together with saw-like cutting blades 33 for the essentially vertical cuts. FIG. 3 shows a close-up view of the mechanism producing the reciprocating motion 35. As described above, the output of the motor 25 shaft is attached to an offset track roller 27. This fits into a slot in the slotted slider 29 which then reciprocates side-to-side as indicated 35. The slotted slider 29 is constrained by the front of the casing 17 (see FIG. 2) to move only in the side-to-side direction. This motion is transmitted to the affixed shaping head 19. The offset track roller 27, being a rolling element bearing, minimizes the wear in the slot. The preferred mechanism is shown; however other physical linkage mechanisms for providing a side-to-side movement can be used. As the cutting head cuts the bone the size of the side-to-side motion is relatively small, typically only a few millimeters in each stroke. A usually rate of motion would be 5-30 cycles per second. In addition, due to the limited space in which the cutting head is located, a small motion is desirable to avoid impingement against soft tissues. It will be appreciated that according to the type and size of femoral (or other) implant component that the bone is being shaped to receive, the cutting heads can be removed and replaced in modular fashion. FIG. 3 also shows the preferred method of attaching the shaping head 19 to the slotted slider 29.

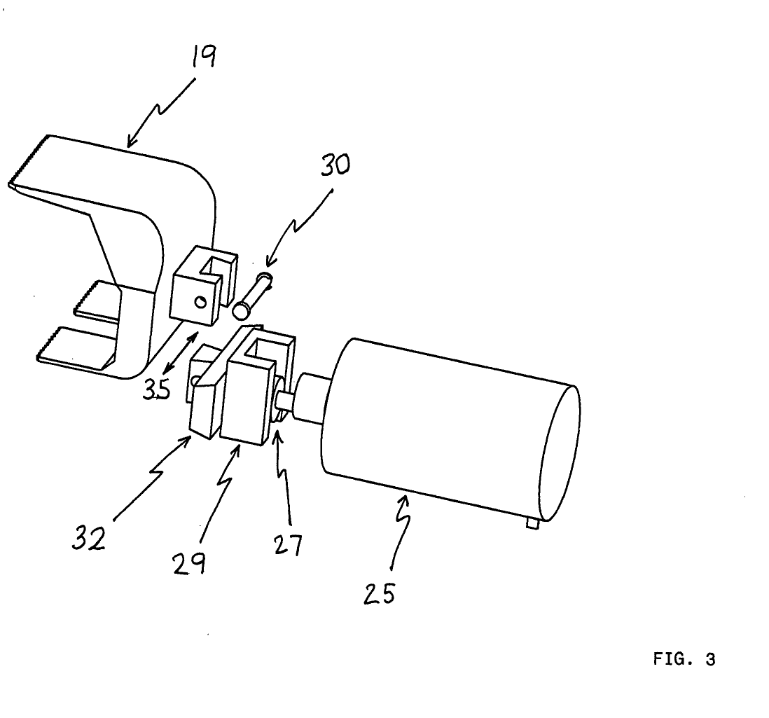

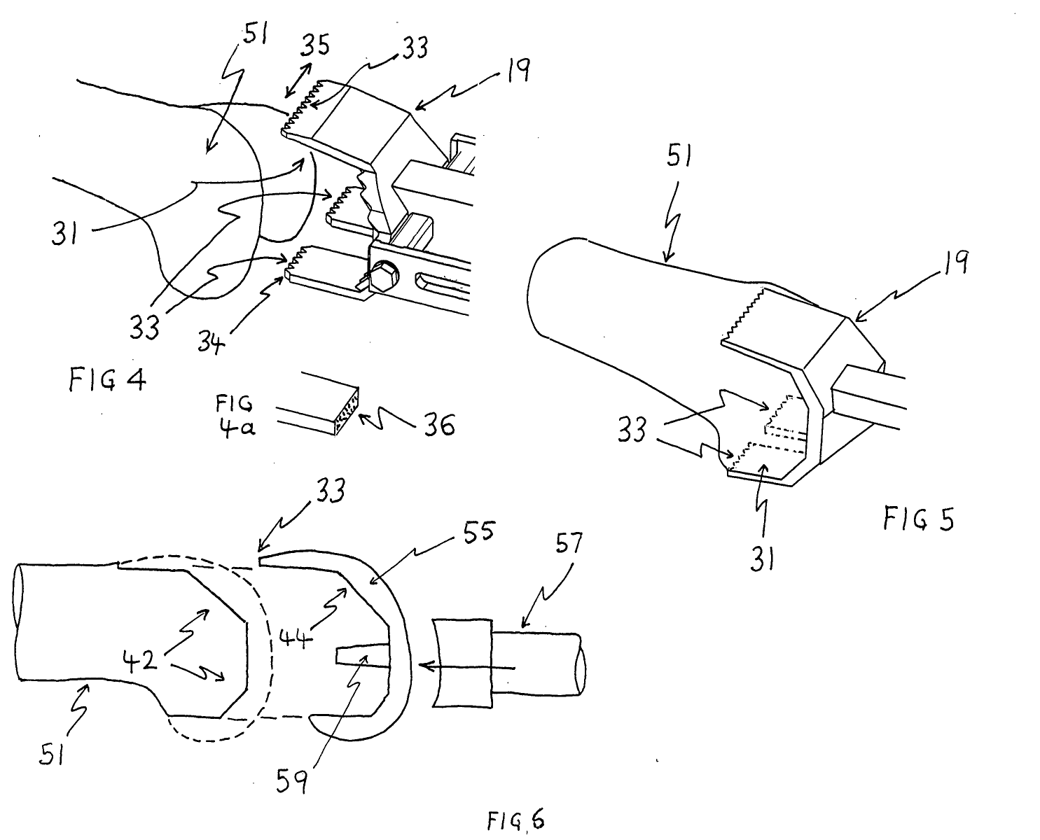

Referring again to FIG. 2, the femoral shaping head 19 is composed of cutting blades 33 and a plurality of shaping surfaces generally at 31. The cutting blades 33 are generally configured to engage the femur as shown in FIG. 4 prior to the engagement of shaping surfaces 31. The cutting blades 33 are linear in design along their length. The blades are generally arranged along the vertical cut directions and have teeth 34 on their ends. The cutting blades 33 are meant to perform the cutting of the substantially vertical surfaces function while the shaping surfaces 31 perform the shaping of the distal end of the femur. Both the cutting blades 33 and the shaping surfaces 31 perform their respective cutting operations through the side-to-side motion (arrow 35). An alternative to the saw blades 33 is to have shaping surfaces in place of the saw teeth as shown at 36 in FIG. 4a, and these surfaces can be at least several millimeters thick extending away from the line of the bone to be cut.

The shaping surfaces shown at 31 are composed of multi-cutting surfaces configured with a metallic rasp-like surface which is intended to grind away the bone surface when the device is applied to the bone and reciprocated. Alternatively, the surfaces may be made from a ceramic material, or other material approximating sand paper or a diamond grinding surface found in the general cutting industry. The specific surface 31 is configured to grind the bone surfaces in such a manner that the resulting bone surface of the femur and/or tibia are precisely shaped to receive the replacement component with little additional work. In cases where there is considerable articular cartilage remaining on the bone surfaces, the grinding may not be so efficient. In this situation, it may be an advantage to carry out a rough cut first using a standard reciprocating saw.

The critical relationships of the device and the implant component are indicated in a comparison of FIGS. 4, 5 and 6. The cutting and shaping device of the present invention is shown in FIG. 4 in relationship with the distal femoral bone 51. The cutting blades 33 will be the first to engage, followed by the shaping surfaces 31. The shaping head 19 is shown having just performed its cutting and shaping function in FIG. 5. It is seen that the distal end of the femur 51 has taken upon the exact shape 42 of the interior of the shaping head 19, shown in FIG. 6. Once this operation has been completed, FIG. 6, the femoral component 55 is attached to the bone end 51 with the aid of a standard impact or 57. Surfaces 42 and 44 match exactly. To fit certain femoral components it may be necessary to carry out extra drilling operations for pegs 59 or other fixation augmentation features.

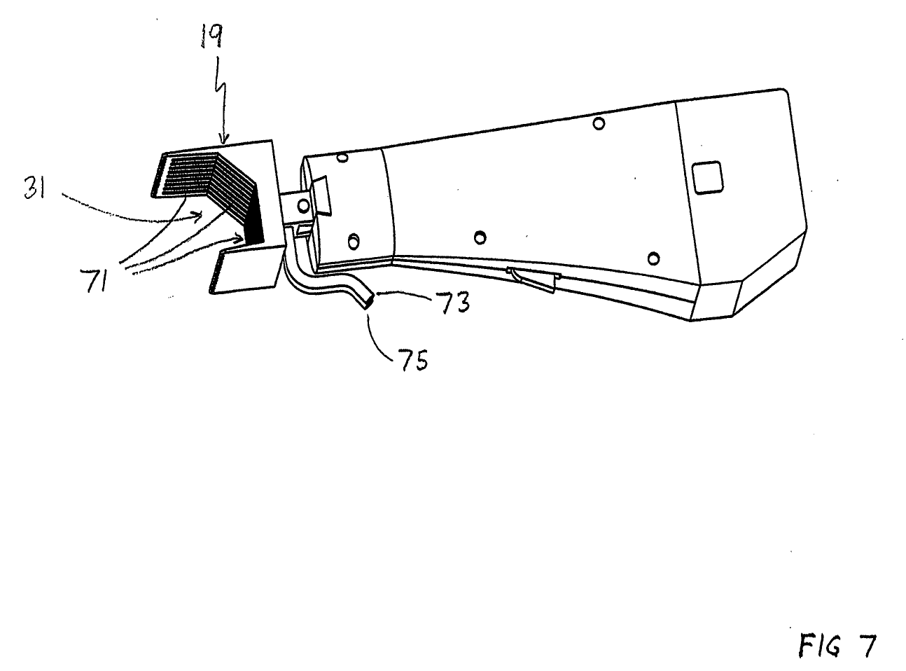

An additional feature of the shaping head and its attachment to the device is shown in FIG. 7. The shaping surfaces 31 have a series of slots or channels 71 cut in them and through at least a portion of them to create apertures through which fluid is supplied and whereby fluids and bone fragments and particles may be carried away. Typically cooling fluid, such as distilled water, or saline will be supplied to the cutting head 19 by means of a tube 73. The fluid will emerge from the aforementioned slots 71 in shaping surfaces 31. Suction will be applied through tube 75 through which fluid and debris are sucked away from some of the slots 71.

As shown in the above figure, the shaping and cutting surfaces, regardless of the specific material of composition, are angularly arranged with respect to each other and positioned to cut and shape multiple angular surfaces simultaneously. As suggested later, the device may be oriented for its cutting and shaping function by hand, through the use of known position navigation devices, electronic positioning, or robotic machine orienting devices known within the medical arts profession. Alternatively, the device of the present invention may be moved and directed in its cutting and shaping function into engagement with the bone cut and shaped by classic bone alignment guides such as intramedullary or extramedullary rods. However, regardless of the manner in which the device according to the invention is positioned, the single cutting and shaping function performed by the device results in a more accurate fit of the components than that achieved by devices which perform a series of singular, but successive cuts to the bone. Therefore, the specific size of the shaping head as well as the specific angular relationship of shaping surfaces 31 to each other and to the cutting blades 33 is critical to achieving the fit and accuracy of the bone to the implant components described above. The cutting and shaping components of the shaping head 19 will vary in their arrangement depending upon the specific knee replacement model.

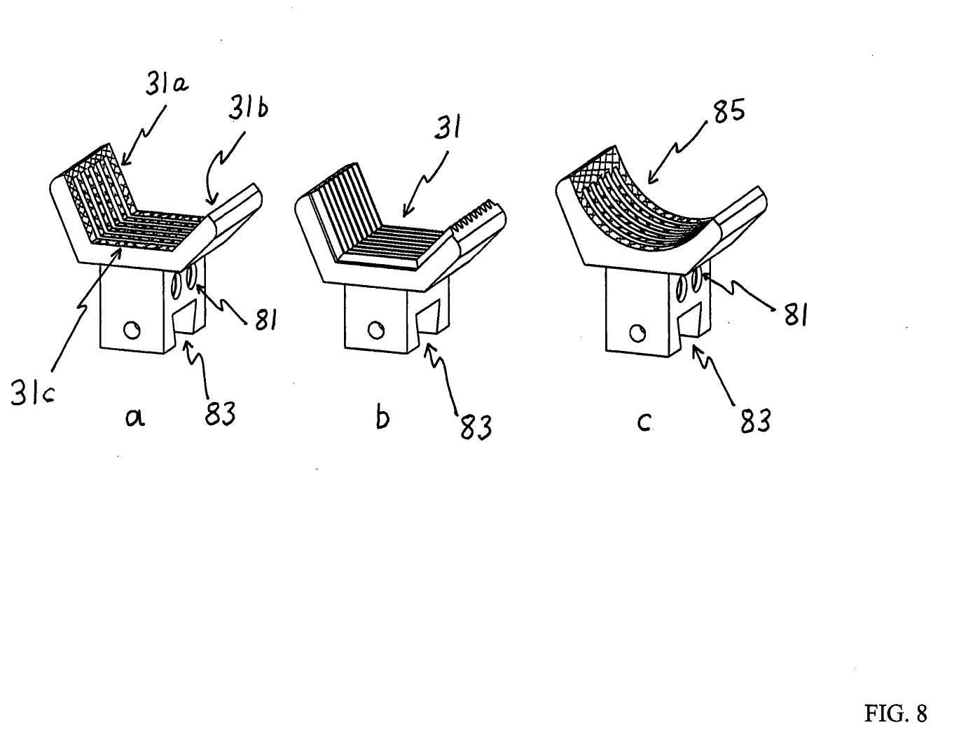

FIG. 8 shows alternative embodiments of the present invention in which the shaping head is shaped for a unicompartmental femoral component. In FIG. 8a, the shaping head is shaped for a conventional unicompartmental femoral component with shaping surfaces 31a, 31b and 31c. Surfaces 31a and 31b are at angles to the base surface 31c corresponding to the implant being used. Cooling and suction holes are shown 81 which provide connection for tubes 73 and 75 shown in FIG. 7. A dovetail slot 83 is shown by which the shaping head is attached to the slotted slider 29 (see FIG. 3) and guided along its side-to-side motion. The shaping surfaces 31a, 31b and 31c have a series of slots or channels cut in them to facilitate cooling and suction as described earlier. FIG. 8b has shaping surfaces consisting of cutting edges having elongated cutting teeth. This shaping head is shown without the built-in suction and cooling facility. The cutting head shown in FIG. 8c is intended for use in operations dedicated to bone preservation such as a curved unicompartmental component. In this alternative embodiment it is important that proper registration and alignment be achieved in order to accurately achieve the proper curvature along the entire bone surface to be shaped. Due to the integrated full curvature of the shaping surface 85, it is easier to achieve the desired complementary shape of the curved bone preservation component than it is using conventional devices. The entire surface is shaped simultaneously, rather than by sequential grinding using burrs for example to fit the concave shape of the component. The result is more accuracy and precision in the interface between the component and the bone surface of the femur. An advantage of curved components as shown in FIG. 8c is that the strongest bone, which is near the surface, is preserved, thus enhancing the strength of the fixation and the durability of the component-bone interface. A further advantage is that if revision is needed at a later time, there is greater preservation of bone stock. FIG. 8c shows a curved surface of approximately 130 degrees. However, it is understood that the surface could be any size, as much as 180 degrees, depending on the design of the implant. Also the curved surface may be a single cylindrical surface or may be a partially spherical surface or a combination of straight, cylindrical and spherical surfaces. The final shape on the bone surface would take into account the side-to-side motion of the cutting head.

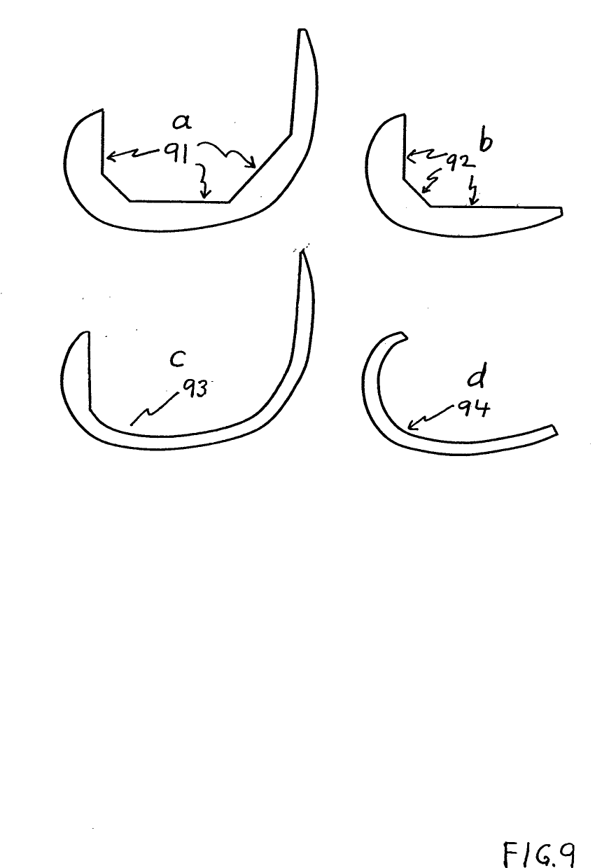

FIG. 9 shows cross section examples of alternate forms of femoral and tibial components (implants) used with the present invention. FIG. 9a shows the side view of a conventional femoral (implant) component of a total knee replacement, having surfaces 91. Likewise, FIG. 9b shows a conventional unicompartmental femoral component having surfaces 92. FIGS. 9c and 9d show corresponding curved components, having curved surfaces 93 and 94 with the advantage of bone preservation and other advantages described above.

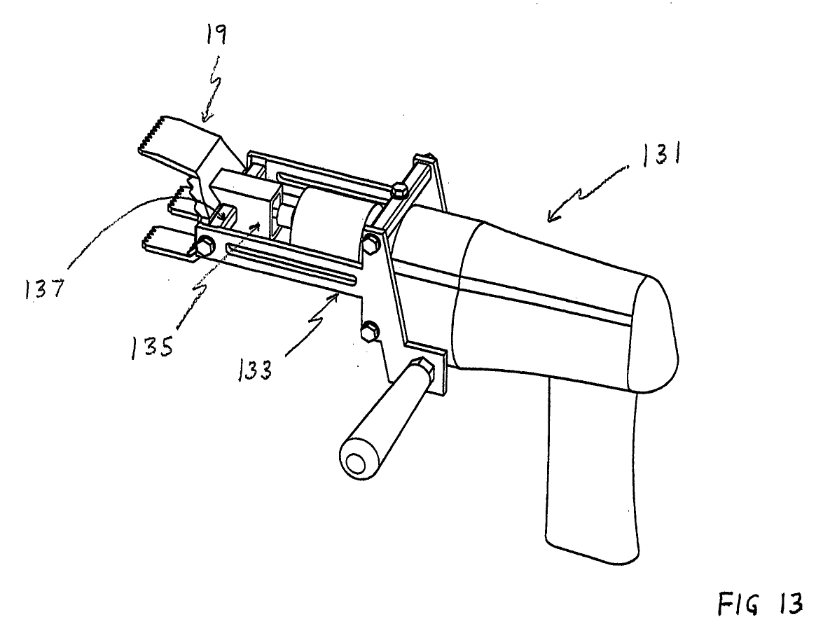

FIGS. 10-12 show apparatus for alignment of bone cuts using the apparatus of this invention. In this case an alternate form of the apparatus is used, further described in FIG. 13. In FIG. 10, the apparatus is shown with an external rod 103 rigidly attached to the framework of the apparatus 105. The rod 103 is maintained by the surgeon in a parallel position to the axis of the bone being cut, in this case the femur 101. The advantage of this method is ease in use, but the disadvantage is that the long axis of the femur has to be estimated visually and there may be an error of a few degrees. An alternative is to direct the rod to the center of the femoral head which is marked on the surgical drapes and which can be determined by palpation or radiography. FIG. 11 shows a similar method as in FIG. 10, but in this case an intramedullary rod 107 is inserted into the femur, and extends outwardly 109 from the distal end of the femur 101, and through an opening in the cutting head. The head is thus kept in alignment during the cutting of the surfaces on the femur. The advantage of this method is that an intramedullary rod provides alignment in both frontal and sagittal planes, and for an appropriately designed rod, it is accurately in alignment with the axis of the femur. The disadvantages of an intramedullary rod are that it is slightly invasive to use and it introduces extra complexity for the apparatus design to accommodate the rod.

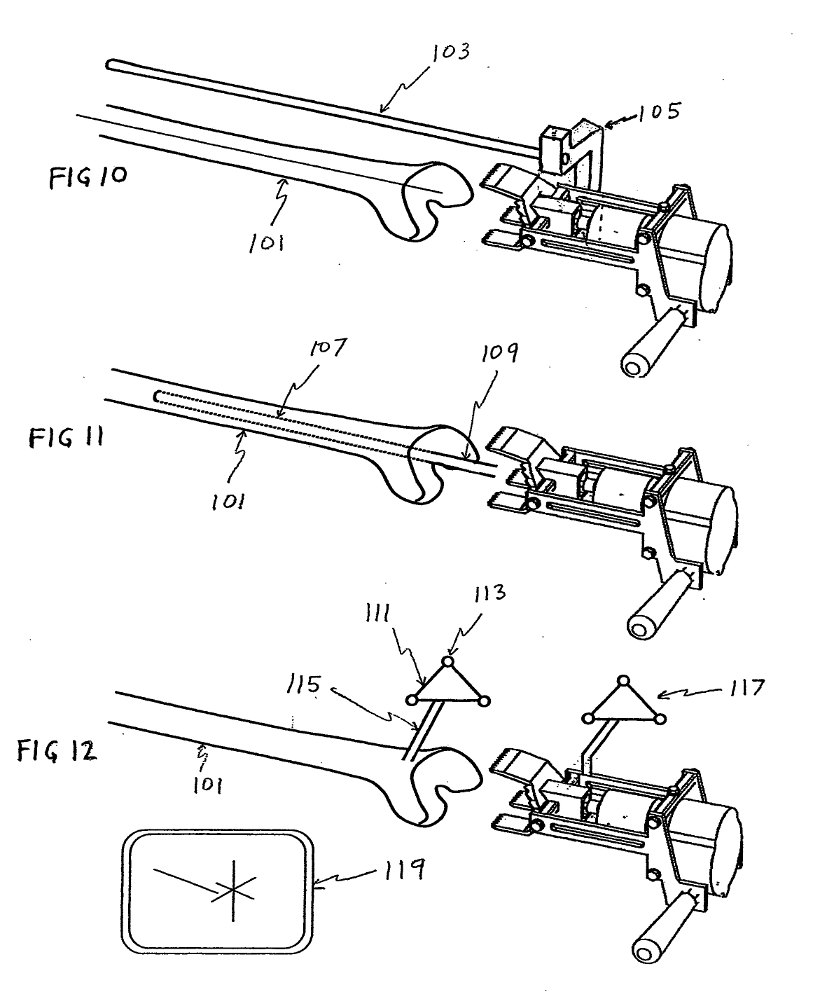

In FIG. 12, a triangular member 111 with reference balls 113 is attached into the femur by a screwed rod 115. A similar triangular member 117 is attached to the framework of the apparatus 105 (see FIG. 10). A camera system with two or three vision points, together with associated computer software, tracks the coordination of the three balls on each triangle and determines the 3-dimensional orientation of the femur and the cutting head apparatus, and hence the relative position of each. Computer models of the bones and cutting tools are moved with these orientations and depicted in their relative positions on the computer screen 119. Feedback to the surgeon is provided on a computer screen 119 or other means. This type of system is called a navigation system, and is well-known in orthopaedics today.

FIG. 13 shows a form of the embodiment whereby an existing type of drill 131 can be used to drive the shaping head. Such drills 131 are commonly used in orthopaedics. An external fixture 133 is rigidly fixed to the drill 131. The mechanism 135 for converting the rotating motion of the drill 131 to side-to-side oscillatory motion is similar to that described in FIG. 3. In this present case, the slotted slider is free to slide side-to-side along rectangular bar 137. To reduce friction and wear between the slotted slider and the rectangular bar, their interface areas may be lined with teflon or ultra-high molecular weight polyethylene, or similar materials. As the shaft rotates, the offset track roller causes the slider to reciprocate side-to-side along bar 137. The cutting head 19 is rigidly attached to the slider and so moves with it. This configuration is shown to illustrate that there are different possibilities for driving the shaping head, using a specially designed driver, or using an existing power source.

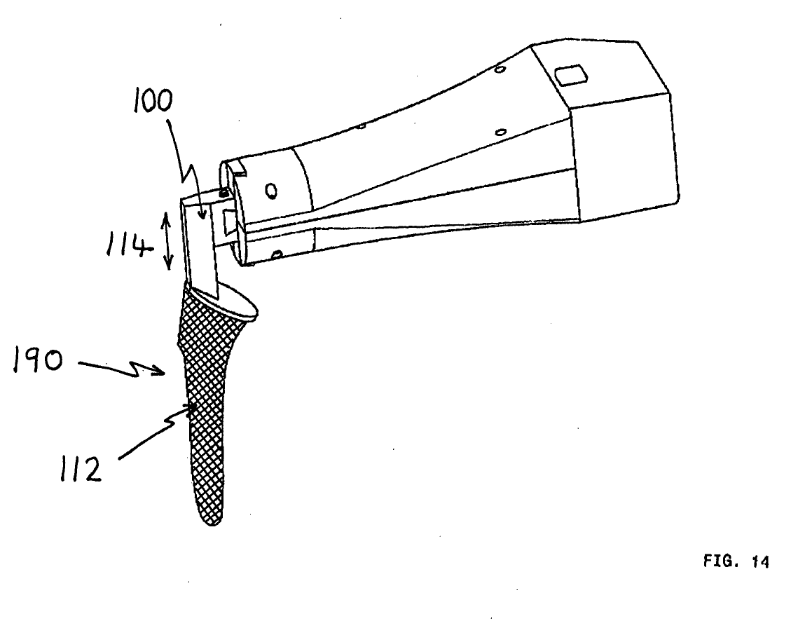

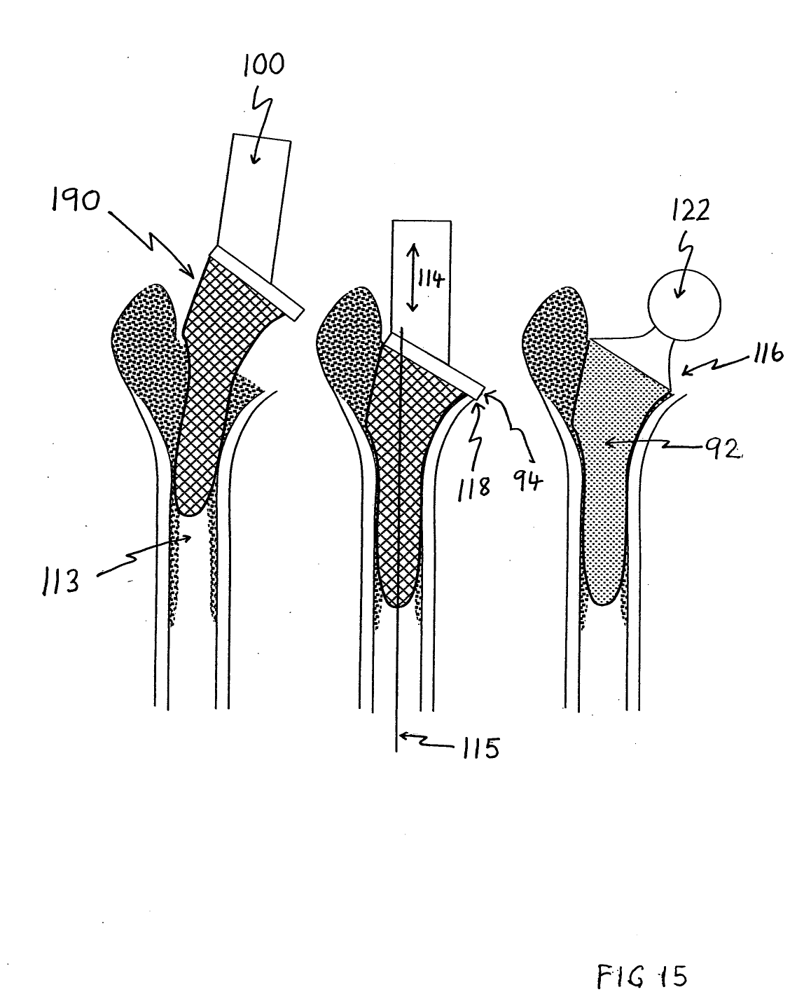

FIG. 14 shows a shaping head which cuts a cavity inside the femur or other bone to insert a prosthesis. Part 100 of the shaping head 190 is inserted in the power unit similar to that shown in FIGS. 1, 2 and 3. The shaping head is reciprocated up and down as shown by the arrow 114 to cut the bone by a rasping and vibrating motion. There are vertical or cross hatched cutting surfaces 112 along the length of the shaping head 190. In FIG. 15, the shaping head 190 is introduced into the femoral cavity 113 and removes primarily cancellous bone. FIGS. 15a and 15b show the cutting head being introduced into the cancellous bone. As the shaping head 190 is advanced, through the cancellous bone, gradually the hard cortical bone is engaged, which then has a substantial self-aligning effect of directing the shaping head down the long axis of the femur 115. The position of final seating is indicated by the collar 94 locating on the cut surface of the bone 118. The shaping head 190 is then removed and the actual implant 116 is impacted into place (FIG. 15c). The implant typically has shaft 92 and femoral head 122. Cooling and suction can be provided during the cutting procedure in the same manner as previously described.

By the foregoing, there has been described different variations of a device for enabling a surgeon to cut and shape the distal femoral and proximal tibial bones for replacement knee surgery. The cutting and shaping is performed using a reciprocating cutting head which is configured to be complementary to the shape of the replacement component. Accordingly, the shape of the cutting head may take a multi-faceted shape, a curved shape, or a flat shape depending upon the shape of the replacement component. This results in increased accuracy of the bone cut, better alignment of the replacement component to the bone, and increased accuracy in alignment of the axes of the two bones in knee replacement surgery. It also results in a reduction in the time required for the procedure.

It will be apparent to those skilled in the art that various additions, substitutions, modifications and omissions can be made to this device and its various embodiments without departing from the scope or spirit of the invention. Thus, it is intended that the present invention covers the additions, substitutions, modifications and omissions provided they come within the scope of the appended claims and their equivalents.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. A bone cutting and shaping device for enabling a surgeon to cut and shape bones in knee replacement, said devise comprising: (a) an assembly extending to the shaping head and configured to transfer motion from a source of reciprocating motion to said shaping head; and (b) an aligning device for permitting the cutting, orienting, and positioning of the cutting head in proper alignment with the bone to be cut and shaped.

18. The cutting and shaping device of claim 17 wherein the shaping head is configured with a concave shape for cutting and shaping a bone to have a resultant convex shape to comport with a concave replacement component.

19. The cutting and shaping device of claim 17 wherein the shaping head is configured with a flat shape for cutting and shaping a bone to a flat shape to comport with a flat replacement component.

20. The cutting and shaping device of claim 17 wherein the aligning device comprises an aligning rod.

21. The cutting and shaping device of claim 17 wherein the aligning device comprises an intramedullary rod for insertion into the bone.

22. The cutting and shaping device of claim 17 wherein the aligning device includes devices for aligning using computer vision-based cameras and targets.

23. (canceled)

24. (canceled)
